(19) United States
(12) Patent Application Publication
(10) Pub. No.: US 2024/0279568 A1
(43) Pub. Date: Aug. 22, 2024

(54) USE OF NEW COMBINATION OF SOLVENTS FOR FRAGRANCES

(71) Applicant: RHODIA BRASIL S.A., São Paulo City (BR)

(72) Inventors: Sergio Martins, Campinas (BR); Caio Cesar Silveira Molinari, Princeton, NJ (US); Laura Viviana Angarita Pinzón, Campinas (BR); Vincent Enaux, Princeton, NJ (US)

(73) Assignee: RHODIA BRASIL S.A., São Paulo City (BR)

(21) Appl. No.: 18/562,714

(22) PCT Filed: May 4, 2022
(86) PCT No.: PCT/IB2022/054110

§ 371 Date: Nov. 20, 2023

(30) Foreign Application Priority Data
May 20, 2021 (WO) PCT/IB2021/054379

(51) Int. Cl. C11B 9/00; A61L 9/03; A61L 9/12; A61L 101/34; A61L 101/38; C11C 5/00

(52) U.S. Cl. CPC C11B 9/0076; A61L 9/037; A61L 9/127; C11B 9/0015; C11C 5/002; A61L 2101/34; A61L 2101/38

(57) ABSTRACT

The present invention relates to fragrances compositions. The present invention, more specifically, relates to the use of a blend of 1,3-butanediol and a glycerol ketal derivative for fragrance compositions. The present invention also discloses fragrance compositions using such a blend to provide controlled evaporation profile.

This application claims priority filed on May 20, 2021 in INTERNATIONAL PROCEDURE with Nr IB2021/054379, the whole content of this application being incorporated herein by reference for all purposes.

### ABSTRACT

The present invention relates to the use of a blend of 1,3-butanediol and a glycerol ketal derivative, in fragrance compositions. The present invention also discloses fragrance compositions using such a blend.

### BACKGROUND

Use of products containing fragrances are part of daily life, including the exposure to fragrances from products that are used to scent the air, such as air fresheners and fragranced candles. Air care devices are constantly facing problems, mainly related to the uncontrolled “distillation” of fragrances components of differing volatilities, which results in a variable fragrance perception over time. The constant evaporation rate and consequently the uniform fragrance evaporation profile, are reached with the use of solvents or carriers in fragrances.

Many materials are used as carriers in air care devices including glycol ethers, isoparaffinic solvents (e.g., Isopar® solvents commercially available from ExxonMobil) and various branched esters. Between glycol ethers, the use of Dowanol DPMA (dipropylene glycol methyl ether acetate) and Dowanol TPM (tripropylene glycol methyl ether) in fragrance composition is known from U.S. Pat. No. 8,603,963. However, these materials come from petrochemical sources and also present a non-pleasure odor.

Fragrances are available in a wide variety and with differences in odor intensity. The odor of carriers or solvents play an important characteristic when considering its use in air care devices as an ideal carrier would be suitable for use with as many fragrances as possible, avoiding any competition in terms of odor.

In spite of the widespread use and exposure of fragrances, the emerging concern of sustainability, low environmental impact, renewable resources and green chemistry are new principles that are guiding the development of the next generation of products, such as solvents for fragrance applications. Such solvents present competitiveness, very good performance in the application, and additionally low toxicity to humans and environment, which meet the three pillars of sustainability (economical, environmental and social).

Alkyl-4-hydroxymethyl-1,3-dioxolane molecules are known to be used in many different applications like fragrances, coatings, paints, cleaning compositions, actives solubilization, because of their good solubilization properties, good physical-chemical properties, low odor and good HSE (Health, Safety and Environment) profile, that can replace products from petrochemical sources. Usually, for air care devices, such molecules are used with petrochemical source materials in order to achieve good evaporation performance. However, the unpleasant odor of the latter makes the handling of the mixture into the final air care fragrance composition to take into account the odor of the mixture and fragrance to be used, in addition to only the evaporation performance.

On the same way, glycols are also attractive from the sustainability point of view and are known to be used in fragrances compositions, mainly as alcohol substitutions and particularly for skin use compositions as described in U.S. Pat. No. 5,677,353 and EP3375430.

Besides the use of a combination of Alkyl-4-hydroxymethyl-1,3-dioxolane and glycol is known for fragrances from US2020306152, those molecules have never been used in combination notably in fragrance compositions for air devices directed for liquid electrical air fresheners, such as electrical plug-in devices and wicking devices as reed diffusers or aromatic candles. Particularly for electrical plug-ins recently developed, the high temperature implemented in devices are considered a challenge in the development of new possibilities for solvent combinations, especially sustainable, which are compatible with the required evaporation profile.

Therefore, there is still a need to provide a sustainable and odorless combination of solvents or carriers able to be used in fragrances compositions and which provides constant, consistent and adequate evaporation of fragrances over time, particularly for air devices directed to fresheners.

### BRIEF DESCRIPTION OF THE INVENTION

The present invention thus provides the use of a blend comprising 1,3-butanediol and at least one glycerol ketal derivative of formula I below:

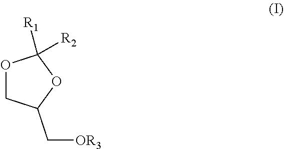

wherein
R₁ and R₂, independently from one another, are selected in the group consisting of: a linear or branched C1-C12 alkyl, a C4-C12 cycloalkyl or an aryl;
R₃ is H, a linear or branched alkyl, a cycloalkyl or a —C(═O)R₄ group, with R₄ being a linear or branched C1-C4 alkyl or a C5-C6 cycloalkyl;
in which the weight ratio glycerol ketal derivative/1,3-butanediol is at least 1;
as solvent or carrier in fragrance compositions.

Indeed, it has been surprisingly found that the use of a blend containing a glycol, notably 1,3-butanediol, and a glycerol ketal derivative in amounts having the weight ratio glycerol ketal derivative/1,3-butanediol of at least 1 in fragrances compositions allow obtaining not only an odorless blend but also a constant and appropriate profile of evaporation of fragrances, particularly for electrical plug-in devices, reed diffusers and aromatic candles. Consequently, the combination of petroleum derived solvents to achieve ideal performance is avoided and the handling of the final air care composition is simplified as the odor intensity of carriers may not need to be considered.

For the purpose of the present invention, an ideal evaporation performance is achieved when there is constant and consistent evaporation over a time period around 30 days.

A further advantage is that the blend ingredients are bio-sourced.

The present invention also aims a fragrance composition comprising the blend as defined above to be used in air care devices, the air care device being an electrical device, a wicking device or an aromatic candle.

Then, another object of the present invention is a fragrance composition comprising a blend of 1,3-butanediol and at least one glycerol ketal derivative of formula I below:

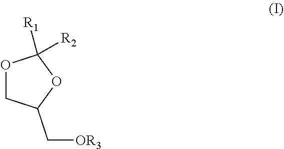

wherein
R1 and R2, independently from one another, are selected in the group consisting of a linear or branched C1-C12 alkyl, a C4-C12 cycloalkyl or an aryl;
R3 is H, a linear or branched alkyl, a cycloalkyl or a —C(═O)R4 group, with R4 being a linear or branched C1-C4 alkyl or a C5-C6 cycloalkyl,
in which the weight ratio glycerol ketal derivative/1,3-butanediol is at least 1.

### DETAILED DESCRIPTION OF THE INVENTION

### Definitions

As used herein, the term “glycerol ketal derivative” refers to compounds that are derived from a similar compound and consequently, have similar structure.

As used herein, the term “1,3-butanediol” refers to any kind of presentation of the compound whether it is a racemic mixture or an isolated enantiomer, for instance “(R)-(−)-1,3 butanediol” or “(S)-(+)-1,3 butanediol”.

As used herein, the term “electrical liquid air freshener device” refers to device or system that includes an electrical or battery source of energy. The term “electrical liquid air freshener device” includes heated liquid wick systems, piezoelectrical spraying systems, electrospray devices and Venturi devices, as well as devices that are powered by solar or other alternative forms of energy.

As used herein, the term “wick device” refers to a device or system that doesn't include an electrical or battery source of energy. The term “reed diffuser” is applied for wick type devices that use the principle of capillarity of the formula that evaporates through the sticks.

All percentages and ratios as used herein are by weight of the total composition, unless otherwise specified. All such weights as they pertain to listed ingredients are based on the active level and, therefore, do not include solvents or by-products that may be included in commercially available materials, unless otherwise specified.

Terms “a” and “an” should be considered as a singular or a generic plural all along the present specification, except otherwise specified.

As used herein, “ambient” evaporation is a type of vaporization that occurs naturally in a mixture of liquids through any kind of environment.

Should the disclosure of any patents, patent applications, and publications which are incorporated herein by reference conflict with the description of the present application to the extent that it may render a term unclear, the present description shall take precedence.

### Glycerol Ketal Derivative of Formula I

The present invention concerns the use of a blend comprising at least one glycerol ketal derivative of formula I as described above.

In a preferred embodiment, R1 and R2, independently from one another, are selected in the group consisting of: methyl, ethyl, isopropyl, n-propyl, isobutyl, n-butyl, tert-butyl, n-pentyl, cyclopentyl, cyclohexyl or phenyl.

Advantageously, in formula I above R3 is H or a —C(═O)R4 group, with R4 being methyl, ethyl, isopropyl, n-propyl, isobutyl, n-butyl or tert-butyl. One preferred embodiment is when R1 and R2 are methyl and R3 is H. In this case, the compound is commercially available, for example under the name Augeo® Clean Multi, Augeo® SL191 or Solketal. This compound can be synthesized by reaction between glycerol and acetone, under well-known classical conditions.

Glycerol can be obtained as a coproduct from biodiesel production during the transesterification of triglycerides.

### Glycol

The present invention also concerns to the use of a blend comprising one glycol, notably 1,3-butanediol, as described above.

Conventionally, this glycol has usually been described as compounded in cosmetics such as perfume, lotion, cleanser, milky lotion; home care products such as liquid dishwashing products, toilet cleaners or products for cleaning various surfaces and air care spray fresheners. It has not been used in air devices such as those of the present invention.

In a particular preferred embodiment, the weight ratio between 2,2-dimethyl-1,3-dioxolane-4-methanol and 1,3-butanediol is from 70:30 to 50:50.

### Air Care Devices

The present invention also aims an air care device, the air care device being an electrical device, wicking device or aromatic candles.

The term “air care device” includes any suitable surface that allows for at least some evaporation of volatile materials. Any suitable air care device having any suitable size, shape, form, or configuration can be used. Suitable air care devices can be made from any suitable material, including but not limited to: natural materials, man-made materials, fibrous materials, non-fibrous materials, porous materials, non-porous materials, and combinations thereof. In certain embodiments, the air care devices used herein are flameless in character and include any device used for dispensing any type of volatile material (e.g. liquids) into the atmosphere (such as fragrance, deodorant, disinfectant or insecticide active agent). In certain non-limiting embodiments, a typical air care device utilizes a combination of a stick, wick, gel, and/or porous surface, and an emanating region to dispense a volatile liquid from a liquid fluid reservoir.

In one embodiment, the air care device is an electrical liquid air freshener device. As noted above, the term “electrical liquid air freshener device” or “liquid electrical air freshener” refers to device or system that includes an electrical or battery operated source of energy which includes heated liquid wick delivery systems, piezoelectrical spraying systems, electrospray devices or Venturi devices. Commercial examples of electrical liquid air freshner devices include, but are not limited to, Glade® PlugIns® Scented oil, sold by SC Johnson & Sons; Air Wick Scented Oils, and Air Wick X-Press® Scented Oils, sold by Reckitt Benckiser; Febreze Noticeables sold by Proctor & Gamble Co., Electric Home Air Fresheners, sold by the Yankee Candle Co.; and Renuzit Scented Oils, sold by Henkel AG.

Advantageously, the air care device can be a liquid electrical air freshener, particularly a plug-in device.

Air care devices (such as, wicking devices) are known for dispensing volatile liquids into the atmosphere, such as fragrance, deodorant, disinfectant or insecticide active agent. A typical air care device utilizes a combination of a wick and emanating region to dispense a volatile liquid from a liquid fluid reservoir. Air care devices are described in U.S. Pat. Nos. 1,994,932; 2,597,195; 2,802,695; 2,804,291; 2,847,976. Ideally, the air care device should require little or no maintenance and should perform in a manner that allows the volatile material to be dispensed at a steady and controlled rate into the designated area while maintaining its emission integrity over the life span of the device.

In another possibility, the wick device is a reed diffuser. As noted above, the term “reed diffuser” refers to device or system that does not include an electrical or battery operated source of energy, but this type of devices uses the principle of capillarity of the formula that evaporates through the sticks. Commercial examples of reed diffuser devices include, but are not limited to Air Wick Scented Oils, sold by Reckitt Benckiser and Renuzit Scented Oils, sold by Henkel.

Preferentially, the wicking device is a reed diffuser.

Alternatively, air care devices are candles. Candles are a key segment of the home fragrance market. Fragrance drives the sale of candles, followed by color and packaging. Consumers are increasingly demanding products that are attractive, economical, safe, and perform well. Typically, most candles are paraffin-based. However, it is also possible to find candles using waxes made from vegetable oil, such as Soybean, coconut, palm kernel oil, beeswax, or blends, for example. A solvent generation adding sustainable value to this market made from renewable sources and having odorless properties is aligned with the market's needs, as mentioned above.

Advantageously, the air care device is an aromatic candle.

### Evaporation Performance

In one embodiment, the use of the blend of the present invention promotes the controlled evaporation rate for use in fragrance composition preferably in an air care device, the air care device being an electrical device, a wicking device or an aromatic candle.

As used above, the controlled evaporation rate is measured as weight of loss of the blend/fragrance, per unit of time (e.g, 0.3-1.5 g/day) over a given time period (e.g., 30 days) is relatively constant over the given time period. In one embodiment, the evaporation rate is relatively constant when the weight of a component loss per unit of time differs by less than +5%, or +10% or +20% over the given time period, for example 30, 40 or 60 days.

Typically, the evaporation rate of the fragrance compositions can be controlled to dispense the fragrance into the surrounding environment over extended periods of time, when electrical plug ins, reed diffusers or aromatic candles are implemented. However, such a controlled dispenser is achieved using a combination of petroleum derived solvents.

It has surprisingly been found that the use of a blend of 1,3-butanediol and at least one glycerol ketal derivative of formula I in fragrance compositions can yield hedonically pleasing, constant and ideal evaporation profiles when used in air care devices of the present invention. It has been proven that combinations using weight ratios of 2,2-dimethyl-1,3-dioxolane-4-methanol/1,3-butanediol from 70:30 to 50:50 allow excellent controlled evaporation results compared to ratios having major amounts of 1,3-butanediol or even compared to blends with glycerol ketal derivative of formula I and other glycols different from 1,3-butanediol. Such blends are able to deliver the content of air care devices above described in an optimal way, constant and over a time period of around 30 days. Notably, such a constant evaporation profile is reached both in electrical plug-ins and during “ambient” evaporation when liquid candles and reed diffusers are implemented.

Advantageously, the blend of the solvents of the present invention is totally sustainable and odorless compared to the usual blend of solvents used in fragrances. A low odor blend allows easily manufacture of fragrances compositions as no competition with fragrances odor occurs. In other words, it is possible to consider the blend as suitable for any kind of fragrances with different odor intensity.

### Fragrances Compositions

The present invention also aims a fragrance composition comprising a blend of 1,3-butanediol and at least one glycerol ketal derivative of formula I below:

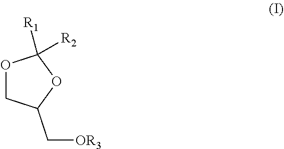

wherein
R1 and R2, independently from one another, are selected in the group consisting of: a linear or branched C1-C12 alkyl, a C4-C12 cycloalkyl or an aryl;
R3 is H, a linear or branched alkyl, a cycloalkyl or a —C(═O)R4 group, with R4 being a linear or branched C1-C4 alkyl or a C5-C6 cycloalkyl,
in which the weight ratio glycerol ketal derivative/1,3-butanediol is at least 1.

All the preferred embodiments detailed before, taken alone or in combination are also applicable to the fragrance composition.

In a preferred embodiment, the fragrance composition comprises from 5 to 90% by weight of the blend, preferably from 5 to 40% by weight based on the total weight of the fragrance composition.

Other details or advantages of the invention will become more clearly apparent in the light of the examples given below.

### BRIEF DESCRIPTION OF THE FIGURE

For a more complete understanding of various embodiments of the described subject matter and its advantages, reference is now made to the following description, taken in conjunction with the accompanying drawings, in which:

FIG. 1 is a comparison evaporation profile between blends A1 to A3 and Ref 1, according to Example 1, measured as the weight loss of the composition during the time.

FIG. 2 is a comparison evaporation profile between blends B1 to B3 and Ref 2, according to Example 1, measured as the weight loss of the composition during the time.

FIG. 3 is a comparison evaporation profile between blends C1 and TPM, according to Example 1, measured as the weight loss of the composition during the time.

FIG. 4 is a qualitative comparison of blends D1 to D4 according to example 2 with a score according to the odor intensity.

FIG. 5 is a qualitative comparison of blends D1 to D4 according to example 2 with a score according to the odor displeasing level.

### EXAMPLES

The present application is further described by means of the examples, presented below, wherein the abbreviations have the usual meaning in the art.

### Methodology to Measure Evaporation Rate and Weight Loss

1. Weight the air care device. Write that value down
2. Prepare the blend of solvent according to the formulation
3. Put the device recipient that will store the blend formulation on the balance. Tare the balance
4. Add to the device recipient the amount of blend needed. Write down the weight added.
5. Tare the balance and weight the whole system. Write that value down
6. Start the test
7. Weight the system 1 to 3 times a week, until you achieve 100% of the system evaporation. Write the values down.
8. Subtract the air care device weight to the whole system weight on each measurement. This will be the amount of blend remaining on the recipient
9. Subtract the initial weight of the blend formulation to the remaining weight. This will be the weight loss of your system
10. Calculate the evaporation rate (g/day) by dividing the weight evaporated by the number of days of the test.

### Example 1

Blends of 1,3-butanediol and Augeo Clean Multi (2,2-dimethyl-1,3-dioxolane-4-methanol) were prepared by simply mixing the components in a recipient. Augeo Clean Multi (2,2-dimethyl-1,3-dioxolane-4-methanol) is commercially available, sold by Rhodia Brasil S.A.

Glycol comparative blends were prepared using 2-Methylpentane-2,4-diol commercially available and sold by Rhodia Brasil S.A and 1,3 propanediol commercially available and obtained from Sigma-Aldrich.

Comparative blends were prepared using commercially-available Dowanol DPMA and Dowanol TPM Glycol Ether sold by DOW in proportions usual for fragrances. These comparative blends are referred as Reference blends.

TABLE 1
Blend of solvent tested

| | A1 | A2 | A3 | Ref. 1 | B1 | B2 | B3 | Ref. 2 | C | Ref. 3 |
|---|---|---|---|---|---|---|---|---|---|---|
| Solvent | % w/w | % w/w | % w/w | % w/w | % w/w | % w/w | % w/w | % w/w | % w/w | % w/w |
| Augeo Clean | 50 | 50 | 50 | — | 70 | 70 | 70 | — | 30 | — |
| Multi | | | | | | | | | | |
| 1,3-Butanediol | 50 | | | — | 30 | — | — | — | 70 | — |
| 2-Methylpentane- | — | 50 | | — | — | 30 | — | — | — | — |
| 2,4-diol | | | | | | | | | | |
| 1,3 propanediol | — | | 50 | — | — | — | 30 | — | — | — |
| Dowanol DPMA | — | | — | 50 | — | — | — | 80 | — | — |
| Dowanol TPM | — | | — | 50 | — | — | — | 20 | — | 100 |

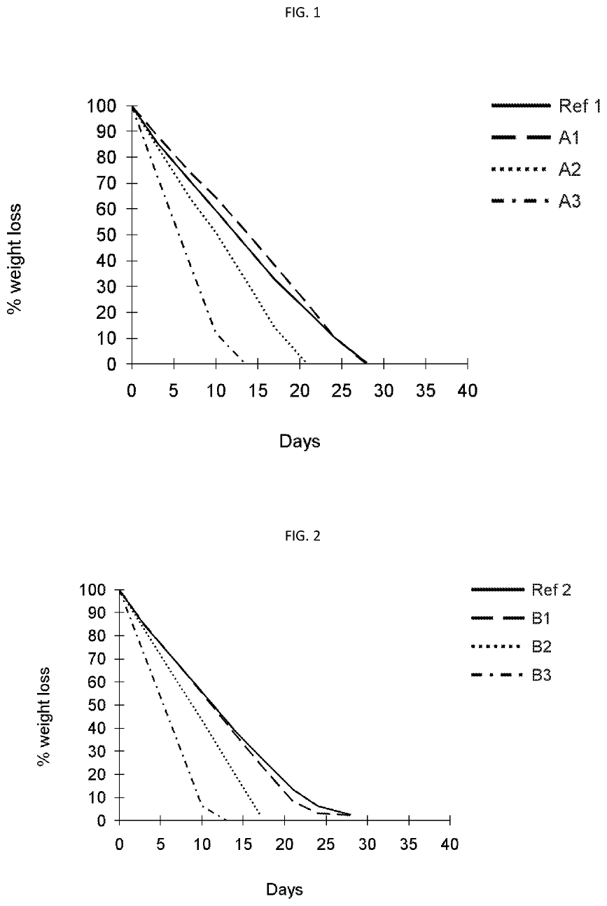

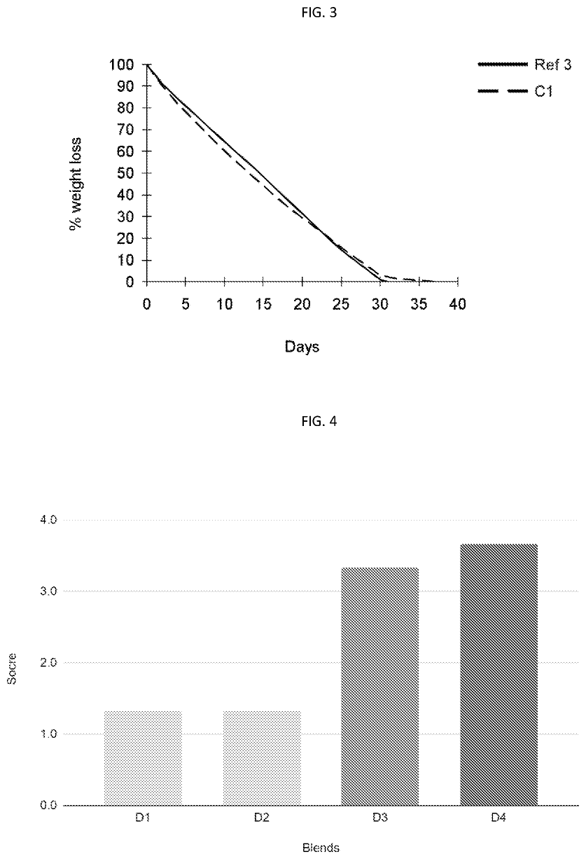

Evaporation profile of blends A1 and B1, according to FIG. 1 and FIG. 2, have demonstrated controlled evaporation performance according to the ideal time release around 30 days compared to commercial blends Ref. 1 and Ref. 2, respectively.

Evaporation profiles of blends A2, A3, B2 and B3 demonstrated poor performance of evaporation, according to FIG. 1 and FIG. 2, showing that glycols different from 1,3-Butanediol did not show an ideal time release to the use in compositions of the present invention.

The blend C has demonstrated performance of evaporation similar to pure Dowanol TPM, which is useless to compositions of the present invention as it has demonstrated such a slow and extended evaporation of the composition.

### Example 2

The blends were submitted to an odor panel test using six participants. The same proportion of solvents were used to evaluate the odor intensity of different blends. All tests were blind, so none of the participants knew which molecules or blends were involved in the test.

Additionally, participants were asked to compare qualitatively all blends and to give a score according to the odor intensity and displeasing level.

The preferred blends were prepared according to example 1:

TABLE 2
Blends of solvents

| | D1 | D2 | D3 | D4 |
|---|---|---|---|---|
| Solvent | % w/w | % w/w | % w/w | % w/w |
| 1,3-Butanediol | 70 | 50 | — | — |
| Augeo Clean Multi | 30 | 50 | — | — |
| Dowanol DPMA | — | — | 70 | 50 |
| Dowanol TPM | — | — | 30 | 50 |

TABLE 3
Blends odor comparison

| | Odor Intensity | Odor displeasing level |
|---|---|---|
| | (more+/less−) | (more+/less−) |
| D3 and D4 | + | + |
| D1 and D2 | − | − |

As shown by Table 3 and FIG. 4 representing the odor intensity from very weak (lowest score) to super strong (highest score), both blends D1 and D2 have demonstrated less intense odor compared to the D3 and D4.

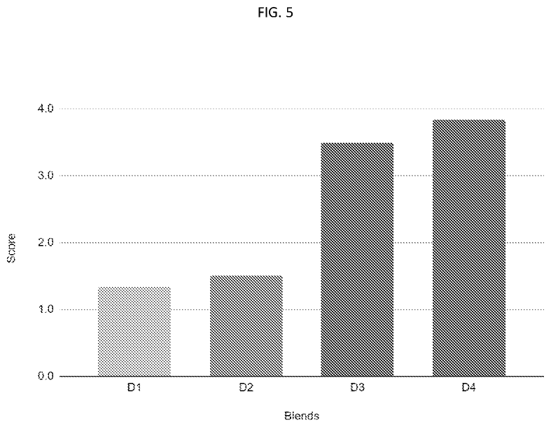

From the qualitative comparison of blends, FIG. 5 representing the odor displeasing level, from non-unpleasant (lowest score) to super unpleasant (highest score), blends D1 and D2 showed less displeasing odor compared to D3 and D4. Indeed, blend D1 has demonstrated even better results in terms of odor intensity compared to the others.

Therefore, the blends of the invention demonstrated not only odorless properties but also a constant and appropriate profile of evaporation of fragrances, allowing better sensorial perception of the fragrance in the environment during evaporation of the composition.

The use of such examples is illustrative only and in no way limits the scope and meaning of the invention or of any exemplified term or phrase used herein. Likewise, the invention is not limited to any particular preferred embodiments described herein. Indeed, many modifications and variations of the invention will be apparent to those skilled in the art upon reading this specification.

It is further to be understood that all values are approximate, and are provided for description.

1. A method for preparing a solvent or carrier in fragrance compositions comprising:
blending 1,3-butanediol and at least one glycerol ketal derivative of formula I below: 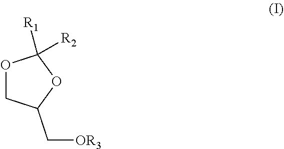
wherein
R₁ and R₂, independently from one another, are selected in the group consisting of: a linear or branched C1-C12 alkyl, a C4-C12 cycloalkyl or an aryl;
R₃ is H, a linear or branched alkyl, a cycloalkyl or a —C(═O)R₄ group, with R₄ being a linear or branched C1-C4 alkyl or a C5-C6 cycloalkyl;
in which a weight ratio of a blend of the glycerol ketal derivative and 1,3-butanediol is at least 1.

2. The method according to claim 1, wherein R₁ and R₂, independently from one another, are selected from the group consisting of methyl, ethyl, isopropyl, n-propyl, isobutyl, n-butyl, tert-butyl, n-pentyl, cyclopentyl, cyclohexyl, or phenyl.

3. The method according to claim 1, wherein R₃ is H or a —C(═O)R₄ group, with R₄ being methyl, ethyl, isopropyl, n-propyl, isobutyl, n-butyl, or tert-butyl.

4. The method according to claim 2, wherein R₁ and R₂ are methyl and R₃ is H.

5. The method according to claim 1, wherein the blend is a mixture of 2,2-dimethyl-1,3-dioxolane-4-methanol and 1,3-butanediol.

6. The method according to claim 5, wherein the weight ratio between 2,2-dimethyl-1,3-dioxolane-4-methanol and 1,3-butanediol is from 70:30 to 50:50.

7. The method according to claim 1, further comprising promoting an evaporation performance of fragrance compositions.

8. A fragrance composition comprising a fragrance and a blend of 1,3-butanediol and at least one glycerol ketal derivative of formula I below: 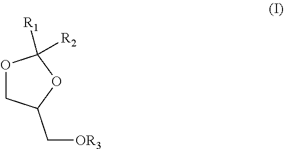
wherein
R₁ and R₂, independently from one another, are selected in the group consisting of a linear or branched C₁-C₁₂ alkyl, a C₄-C₁₂ cycloalkyl or an aryl;
R₃ is H, a linear or branched alkyl, a cycloalkyl or a —C(═O)R₄ group, with R₄ being a linear or branched C₁-C₄ alkyl or a C₅-C₆ cycloalkyl,
in which a weight ratio of a blend of the glycerol ketal derivative and 1,3-butanediol is at least 1.

9. The composition according to claim 8, comprising from 5 to 90% by weight of the blend based on the total weight of the fragrance composition.

10. The composition of claim 9, comprising from 5 to 40% by weight of the blend based on the total weight of the fragrance composition.

11. The composition of claim 8, wherein R₁ and R₂, independently from one another, are selected from the group consisting of methyl, ethyl, isopropyl, n-propyl, isobutyl, n-butyl, tert-butyl, n-pentyl, cyclopentyl, cyclohexyl, or phenyl.

12. The composition of claim 8, wherein R₃ is H or a —C(═O)R₄ group, with R₄ being methyl, ethyl, isopropyl, n-propyl, isobutyl, n-butyl, or tert-butyl.

13. The composition of claim 11, wherein R₁ and R₂ are methyl and R₃ is H.

14. The composition of claim 8, wherein the blend is a mixture of 2,2-dimethyl-1,3-dioxolane-4-methanol and 1,3-butanediol.

15. The composition of claim 14, wherein the weight ratio between 2,2-dimethyl-1,3-dioxolane-4-methanol and 1,3-butanediol is from 70:30 to 50:50.

16. The method of claim 7, further comprising promoting the evaporation performance of fragrance compositions from an electrical air care device, a wicking device, or an aromatic candle.
